(19) United States
(12) Patent Application Publication
(10) Pub. No.: US 2013/0331681 A1
(43) Pub. Date: Dec. 12, 2013

(54) OBJECT INFORMATION ACQUISITION APPARATUS AND PHOTOACOUSTIC PROBE

(71) Applicant: CANON KABUSHIKI KAISHA, Tokyo (JP)

(72) Inventors: Toshinobu Tokita, Kyoto-shi (JP); Masato Yajima, Tokyo (JP)

(21) Appl. No.: 13/912,521

(22) Filed: Jun. 7, 2013

(30) Foreign Application Priority Data
Jun. 11, 2012 (JP) 2012-132132

(51) Int. Cl. A61B 5/00

(52) U.S. Cl. CPC A61B 5/0095
USPC 600/407

(57) ABSTRACT

In an object information acquisition apparatus, an optical system and an acoustic wave detector are closely arranged, and when the acoustic wave detector and an object acoustically contact each other through an acoustic matching member, a light exit surface of the optical system is arranged so that the light exit surface does not directly contact the acoustic matching member.

### BACKGROUND OF THE INVENTION

1. Field of the Invention

The present disclosure relates to an object information acquisition apparatus that irradiates an object with light and acquires object information based on a photoacoustic wave generated in the object.

2. Description of the Related Art

Photoacoustic imaging (PAI) is receiving attention as a method of specifically imaging vascularization (angiogenesis) that is generated by cancer.

PAI is a method of irradiating an object with light (typically, near-infrared rays), causing an acoustic wave detector to detect a photoacoustic wave that is generated from the inside of the object by the irradiation, and forming an image of the inside of the object.

Meanwhile, an initial sound pressure P₀ of the photoacoustic wave generated in the object can be expressed by Expression (1) as follows:

P₀=Γ·μₐ·Φ Expression (1),

where Γ is a Grueneisen coefficient, and is obtained by dividing the product of a volume expansion coefficient β and the square of the sound speed c, by a constant pressure specific heat Cₚ. It is known that Γ is a substantially constant value if an object is determined. Also, μₐ is an absorption coefficient, and Φ is a light quantity value.

Japanese Patent Laid-Open No. 2010-88627 discloses a photoacoustic apparatus that causes an acoustic wave detector to detect a change with time of the sound pressure of a photoacoustic wave, which has propagated through an object, and calculates an initial sound pressure P₀ from the detection result. Also, Japanese Patent Laid-Open No. 2010-88627 discloses that, based on Expression (1), the calculated initial sound pressure P₀ is divided by the Grueneisen coefficient Γ, and hence the product of μₐ and Φ, that is, an optical-energy absorption density is acquired. Further, Japanese Patent Laid-Open No. 2010-88627 discloses that, based on Expression (1), the optical-energy absorption density is divided by the light quantity value Φ, and hence the absorption coefficient μₐ is acquired.

Also, Non-patent Document 1 (S. A. Ermilov et al., “Development of laser optoacoustic and ultrasonic imaging system for breast cancer utilizing handheld array probes,” Photons Plus Ultrasound: Imaging and Sensing 2009, Proc. of SPIE vol. 7177, 2009) discloses a photoacoustic apparatus that uses a photoacoustic probe having integrated optical system and acoustic wave detector. Also, Non-patent Document 1 discloses that an object is irradiated with light that is emitted from the optical system, and the acoustic wave detector detects a photoacoustic wave that is generated by the light irradiation.

### SUMMARY OF THE INVENTION

According to an aspect of the invention, an object information acquisition apparatus includes an optical system configured to guide light that is emitted by a light source, and irradiate an object with the light; and an acoustic wave detector configured to detect a photoacoustic wave that is generated when the object is irradiated with the light, and output a detection signal. The optical system and the acoustic wave detector are closely arranged. When the acoustic wave detector and the object acoustically contact each other through an acoustic matching member, a light exit surface of the optical system is arranged so that the light exit surface does not directly contact the acoustic matching member.

Further features of the present invention will become apparent from the following description of exemplary embodiments with reference to the attached drawings.

### BRIEF DESCRIPTION OF THE DRAWINGS

FIG. 1 is a schematic illustration of a photoacoustic apparatus according to a first embodiment.

FIG. 2 is a cross-sectional view of a photoacoustic probe according to the first embodiment.

FIG. 3 is an illustration for explaining a problem of the photoacoustic probe according to the first embodiment of the invention.

FIG. 4 is a cross-sectional view of a photoacoustic probe according to a second embodiment.

### DESCRIPTION OF THE EMBODIMENTS

Discussed here is a case in which an acoustic matching member, such as gel, is applied to a photoacoustic probe having integrated optical system and acoustic wave detector, which is disclosed in Non-patent Document 1.

When measurement is executed with a photoacoustic probe, an acoustic matching member can be provided between an acoustic wave detector and an object to provide acoustic matching.

However, if the photoacoustic probe has the integrated acoustic wave detector and optical system, when the acoustic wave detector contacts the object through the acoustic matching member, the acoustic matching member may adhere to an exit surface of the optical system.

Therefore, an operator such as a medical doctor or a medical technologist has to remove the acoustic matching member adhering to the optical system by cleaning after use in view of sanitation etc. As mentioned above, the cleaning, which is a troublesome work, is required if the acoustic matching member adheres to the optical system.

Further, the acoustic matching member adhering to the optical system may not be sufficiently removed by the cleaning. In this case, the acoustic matching member in a dry condition may adhere to the exit surface of the optical system, and may change optical properties (refractive index, transmissivity, etc.) of the exit surface of the optical system.

Owing to this, in an object information acquisition apparatus according to an embodiment of the invention, an optical system is arranged so that an acoustic matching member adhering to an exit surface of an optical system is reduced.

Accordingly, a troublesome work of cleaning the acoustic matching member adhering to the exit surface of the optical system can be reduced. Further, likelihood of occurrence of that the dry acoustic matching member adheres to the exit surface of the optical system is reduced, and the object can be irradiated with light with a light intensity distribution close to a desirable light intensity distribution.

It is to be noted that object information according to an embodiment of the invention includes an initial sound pressure of a photoacoustic wave generated in an object or an optical-energy absorption density that is derived from the initial sound pressure, an absorption coefficient, a density of a substance that forms a tissue, a density of the substance, and distributions of these values. Also, the density of a substance is, for example, an oxygen saturation, or a density of oxidized/deoxidized hemoglobin.

The invention is described below with reference to the drawings. Like reference signs are basically applied to like configurations, and redundant description for these configurations is omitted.

### First Embodiment

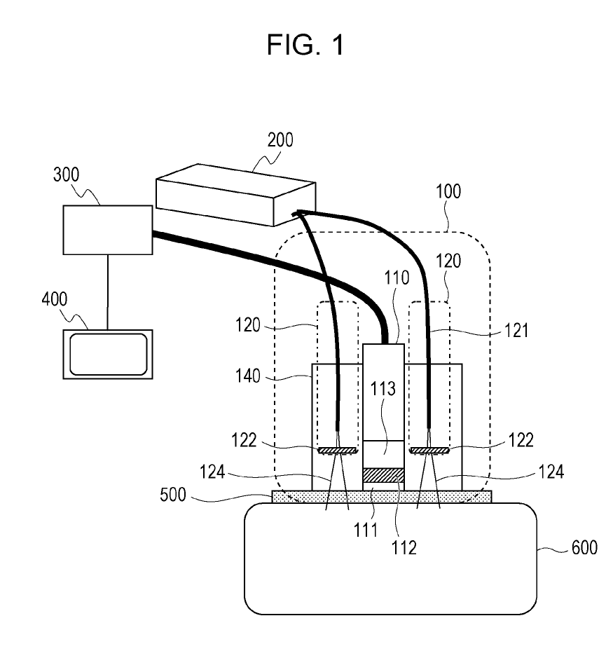

First, a basic structure of a photoacoustic apparatus as an object information acquisition apparatus according to this embodiment is described with reference to FIG. 1. FIG. 1 is a schematic illustration of a photoacoustic apparatus according to this embodiment.

### Basic Configuration

The photoacoustic apparatus according to this embodiment includes a photoacoustic probe 100, a light source 200, a signal processing device 300, and a monitor 400. An acoustic matching member 500 is provided between the photoacoustic probe 100 and an object 600.

The photoacoustic probe 100 according to this embodiment includes an acoustic wave detector 110, an optical system 120, and a casing 140. The acoustic wave detector 110 and the optical system 120 are closely arranged. According to an embodiment of the invention, the state in which “the acoustic wave detector and the optical system are closely arranged” includes a state in which the acoustic wave detector does not directly contact the optical system.

Also, the acoustic wave detector 110 according to this embodiment includes an acoustic matching layer 111, an acoustic wave detection element 112, and a backing member 113. The acoustic matching layer 111 is a layer that provides acoustic matching between the acoustic wave detection element 112 and the acoustic matching member 500. Also, the backing member 113 is a member that controls vibration caused by the photoacoustic wave.

Also, the optical system 120 according to this embodiment includes a bundle fiber 121, and a diffusion plate 122.

Also, the acoustic wave detector 110 acoustically contacts the object 600 through the acoustic matching member 500. Further, at this time, the optical system 120 is arranged so that a light exit surface of the optical system 120 does not directly contact the acoustic matching member 500.

That is, the light exit surface of the optical system 120 is located at an inner side of the casing 140 with respect to a photoacoustic wave detection surface of the acoustic wave detector 110. Also, the light exit surface of the optical system 120 is located farther from the object 600 than the photoacoustic wave detection surface of the acoustic wave detector 110.

Typically, the acoustic matching member 500 is used while an operator applies the acoustic matching member 500 on the surface of the object 600 by a thickness of about 1 mm. Owning to this, the exit surface of the optical system 120 is preferably located at the inner side of the casing 140 by at least 1 mm with respect to the detection surface of the acoustic wave detector 110.

However, the acoustic matching member 500 may not be uniformly applied, and may be applied in a locally bulged manner. Hence, to sufficiently separate the exit surface of the optical system 120 from the acoustic matching member 500, the exit surface of the optical system 120 is more preferably located at the inner side of the casing 140 by at least 5 mm with respect to the detection surface of the acoustic wave detector 110. Accordingly, the likelihood of occurrence of that the acoustic matching member 500 adheres to the exit surface of the optical system 120 can be reliably reduced.

In this embodiment, since the optical system 120 is arranged as described above, the acoustic matching member 500 that adheres to the light exit surface of the optical system 120 can be reduced. Hence, with the photoacoustic apparatus according to this embodiment, the troublesome work of cleaning the acoustic matching member 500 adhering to the exit surface of the optical system 120 can be reduced. Further, likelihood of occurrence of that the solidified acoustic matching member 500 remains on the exit surface of the optical system is reduced, and the object 600 can be irradiated with light with a light intensity distribution close to a desirable light intensity distribution.

According to an embodiment of the invention, a state in which “the object and the acoustic wave detector acoustically contact each other” represents a state in which the acoustic wave detection element of the acoustic wave detector can detect the photoacoustic wave. That is, even though the object and the acoustic wave detector do not directly contact each other, as long as the object and the acoustic wave detector contact each other through the acoustic matching member, it can be said that the object and the acoustic wave detector acoustically contact each other.

Also, according to an embodiment of the invention, “a photoacoustic wave detection surface of the acoustic wave detector” represents an object-side surface of a member that is the closest to the object among members that form the acoustic wave detector, when the object and the acoustic wave detector acoustically contact each other. That is, in this embodiment, “a photoacoustic wave detection surface of the acoustic wave detector 110” represents an object-side surface of the acoustic matching layer 111.

Also, according to an embodiment of the invention, “a light exit surface of the optical system” represents an object-side surface of a member that emits light for irradiation on the object among members that form the optical system, when the object and the acoustic wave detector acoustically contact each other. That is, in this embodiment, “a light exit surface of the optical system 120” represents an object-side surface of the diffusion plate 122.

In this embodiment, the optical system 120 including two propagation optical paths is arranged to sandwich the acoustic wave detector 110. However, an embodiment of the invention is not limited thereto. For example, two acoustic wave detectors 110 may be arranged to sandwich the optical system 120. Alternatively, one acoustic wave detector 110 and one optical system 120 may be closely arranged. Still alternatively, a plurality of acoustic wave detectors 110 and a plurality of optical systems 120 may be alternately arranged.

Also, in this embodiment, the acoustic wave detector and the optical system may be arranged in a desirable manner as long as “the photoacoustic wave detection surface of the acoustic wave detector” and “the light exit surface of the optical system” are arranged in the above-described manner.

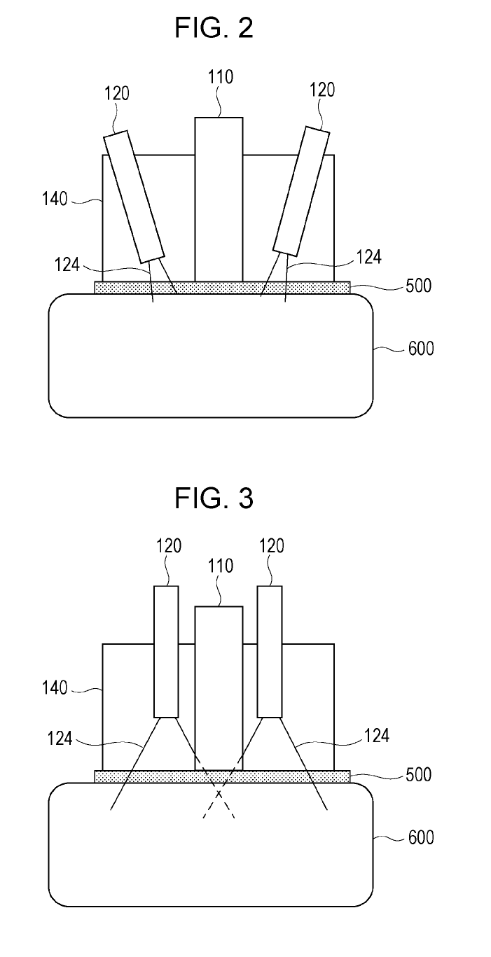

For example, like a photoacoustic probe shown in FIG. 2, “the photoacoustic wave detection surface of the acoustic wave detector 110” and “the light exit surface of the optical system 120” may be nonparallel to each other.

Also, an embodiment of the invention can be applied to a handheld photoacoustic apparatus, and a photoacoustic apparatus having a photoacoustic probe mounted on a robot arm or a stage.

Meanwhile, if the acoustic wave detector and the optical system are arranged as described above, the acoustic wave detector 110 may be irradiated with light 124 that is emitted from the optical system 120 as shown in FIG. 3. In this case, the emitted light 124 is blocked by the acoustic wave detector 110. Hence, the intensity distribution of the light 124 with which the object 600 is irradiated may not become a desirable intensity distribution. Further, if the acoustic wave detector 110 is irradiated with the emitted light 124, a photoacoustic wave may be generated at the acoustic wave detector 110, and the photoacoustic wave may cause a noise of a detection signal.

Owing to this, the acoustic wave detector and the optical system according to an embodiment of the invention are desirably arranged so that the acoustic wave detector is not irradiated with the light emitted from the optical system, with regard to a size of each member and a spread state of the light that is emitted from the optical system.

Also, the member that forms the acoustic wave detector is desirably formed of a material that transmits light with a wavelength emitted from the optical system. For example, an acoustic wave detection element that transmits near-infrared rays may be PZNT (lead, zirconium, niobium, titanium) single crystal.

With the above-described configuration, the object can be irradiated with light with the desirable intensity distribution. Also, the photoacoustic wave that is generated at the acoustic wave detector by the light emitted from the optical system can be reduced.

### Object Information Acquisition Method

Next, an object information acquisition method using the photoacoustic apparatus according to this embodiment is described with reference to FIG. 1.

First, the light 124 emitted by the light source 200 is guided by the bundle fiber 121, is processed by the diffuser 122 to have the desirable light intensity distribution, and then is emitted on the object 600.

Then, the acoustic wave detection element 112 detects the photoacoustic wave that is generated in the object 600 by the irradiation with the light 124 on the object 600, through the acoustic matching member 500 and the acoustic matching layer 111, and outputs a detection signal.

Next, the signal processing device 300 executes signal processing, such as amplification processing or digital conversion processing, on the detection signal output from the acoustic wave detection element 112. Then, image reconstruction is executed for the detection signal after the signal processing, and hence an initial sound pressure distribution is acquired as object information.

Also, the signal processing device 300 calculates a light quantity distribution of the inside of the object, through simulation using a light-propagation Monte Carlo method, a transport equation, an optical diffusion equation, etc., by using an average optical coefficient etc. of the object, based on the desirable light intensity distribution.

Further, the signal processing device 300 acquires an absorption coefficient distribution of the inside of the object, based on the initial sound pressure distribution and the light quantity distribution of the inside of the object, as shown in Expression (1).

The absorption coefficient distribution acquired by the signal processing device 300 is displayed on the monitor 400.

According to this embodiment, since the likelihood of occurrence of that the acoustic matching member 500 adheres to the light exit surface of the optical system 120 can be reduced, the object 600 can be irradiated with the light 124 with the light intensity distribution close to the desirable light intensity distribution.

Hence, in this embodiment, the light quantity distribution obtained by the simulation of the signal processing device 300 becomes close to a light quantity distribution of light with which the object is actually irradiated. Hence, according to this embodiment, quantitativeness of an absorption coefficient distribution that is acquired by using the light quantity distribution obtained by the simulation becomes higher than that of related art.

The signal processing device according to an embodiment of the invention can accurately acquire any object information that is acquired by using the light quantity distribution obtained by the simulation.

A main configuration of the object information acquisition apparatus according to this embodiment is described below.

### Acoustic Wave Detector 110

The acoustic wave detector 110 may have any configuration as long as the acoustic wave detector 110 includes the acoustic wave detection element 112 that can detect the photoacoustic wave.

For example, an acoustic lens or a protection layer of the acoustic wave detection element 112 that collects the photoacoustic wave may be used as part of the configuration of the acoustic wave detector 110, instead of the configuration according to this embodiment.

Also, the acoustic wave detection element 112 may use any acoustic wave detection element, such as a transducer using a piezoelectric phenomenon, a transducer using resonance of light, or a transducer using a change in capacity, as long as the acoustic wave detection element can detect the photoacoustic wave.

In particular, a capacitive transducer (capacitive micromachined ultrasonic transducer, CMUT) is desirable as the acoustic wave detection element 112 because the CMUT has an acoustic impedance close to that of a human body and the CMUT has a reception characteristic in a broad band region.

Further, the acoustic wave detection element 112 can typically have a plurality of one-dimensionally or two-dimensionally arrayed acoustic wave detection elements. Since such multidimensional-array elements are used, photoacoustic waves can be detected simultaneously at a plurality of positions. The detection time can be reduced, and the influence of, for example, vibration of the object, can be reduced.

### Optical System 120

In this embodiment, the optical system 120 is a general name of an optical element that guides the light emitted by the light source 200 to the object 600. That is, the optical system 120 may have any configuration as long as the configuration can guide the light emitted by the light source 200 to the object 600.

For example, the light may be guided to the object 600 by using a mirror or a total reflection prism.

### Casing 140

The casing 140 is an outer box that houses configurations of the photoacoustic probe, such as the acoustic wave detector 110, and the optical system 120. The casing may be also called a housing.

The material of the casing 140 may be, for example, metal, such as aluminum or steel; resin, such as polycarbonate, acryl, or polyacetal; or an inorganic material such as ceramic.

An object-side surface of the casing 140 is open so that the surface does not disturb the irradiation with the light on the object and the detection of the photoacoustic wave from the object. That is, it is that the casing 140 does not surround an area between the object 600 and the optical system 120, and an area between the object 600 and the acoustic wave detector 110.

However, the area between the object 600 and the acoustic wave detector 110 may be surrounded by the casing 140, as long as the casing 140 is formed of a material that can provide acoustic matching between the object 600 and the acoustic wave detector 110.

Also, the casing 140 may be provided between the object 600 and the optical system 120 as long as the casing 140 is formed of a material that transmits the light with which the object is irradiated. However, in this case, according to an embodiment of the invention, a portion of the casing through which the light propagates is treated as the light exit surface of the optical system. The portion of the casing through which the light propagates has to have a profile that is located farther from the acoustic matching member than the photoacoustic wave detection surface of the acoustic wave detector.

Also, if an embodiment of the invention is applied to the handheld photoacoustic apparatus, the casing 140 can have a grip portion to allow an operator to grip the photoacoustic probe.

### Light Source 200

The light source 200 can emit near-infrared rays, for example, with wavelengths in a range from about 600 to 1100 nm. Alternatively, the light source 200 can emit pulsed light with pulses in a range from 5 to 50 nanoseconds.

A laser is desirable as the light source 200 because the laser provides high power. Alternatively, a light emitting diode may be used instead of the laser.

The laser may be any of various lasers, such as a solid-state laser, a gas laser, a fiber laser, a dye laser, and a semiconductor laser.

For example, the light source 200 may be a pulse laser, such as a Nd:YAG laser or an alexandrite laser. Alternatively, a Ti:sapphire laser or an OPO laser that uses a Nd:YAG laser beam as excited light may be used.

### Signal Processing Device 300

The signal processing device 300 can amplify the detection signal that is obtained from the acoustic wave detector 110, and can convert the detection signal from an analog signal to a digital signal.

In this specification, a concept of the “detection signal” includes an analog signal output from the acoustic wave detector 110 and a digital signal that is AD-converted from the analog signal.

Further, the signal processing device 300 acquires an optical-property-value distribution of the inside of the object by the image reconstruction based on the detection signal. The signal processing device 300 typically uses a workstation etc., and processing is executed by software in which image reconstruction processing etc. is previously programmed.

An image reconstruction algorithm may be, for example, reverse projection in time domain or Fourier domain which is generally used for a tomography technique.

If the reconstruction can use a long time, an image reconstruction method such as an inverse problem analysis method by repetition processing may be used.

Also, the optical property distribution of the inside of the object can be formed without the image reconstruction if the acoustic wave detector having the acoustic lens etc. is used. In such a case, the signal processing using the image reconstruction algorithm may not be executed.

Also, if the detection signal obtained from the acoustic wave detector 110 is a plurality of detections signals, the signal processing device 300 desirably simultaneously processes the plurality of signals. Accordingly, a time required until formation of an image can be reduced.

Also, the signal processing device 300 may be formed of separate devices, such as an amplifier, an A/D converter, a field programmable gate array (FPGA) chip, etc.

The processing executed by the signal processing device 300 may be formed as a program that is executed by a computer.

Also, the signal processing device 300 may be provided as part of the configuration of the photoacoustic probe. At this time, a signal processing device provided in the photoacoustic probe may execute part of the signal processing, and a signal processing device provided outside the photoacoustic probe may execute the residual signal processing.

### Monitor 400

The monitor 400 is a device that displays the optical property value output from the signal processing device 300. The monitor 400 is typically a liquid crystal display. The monitor 400 may be provided separately from the object information acquisition apparatus of an embodiment of the invention.

### Acoustic Matching Member 500

The acoustic matching member 500 is described below although this is not part of the object information acquisition apparatus of an embodiment of the invention. The acoustic matching member 500 is a member that is provided between the object 600 and the acoustic wave detector 110 and provides acoustic matching between the object 600 and the acoustic wave detector 110.

The acoustic matching member 500 may be typically gel that is made of, for example, water, oil, or alcohol.

### Object 600

The object 600 is described below although this is not part of the object information acquisition apparatus of an embodiment of the invention. A main purpose of the object information acquisition apparatus according to this embodiment is diagnosis for a malignancy and a vascular disease of a human and an animal, and follow-up of a chemotherapy treatment. Hence, for the object 600, a subject portion of diagnosis, such as a breast, a finger, an arm, and a leg of a human body or an animal body, may be expected.

For example, if the object 600 is a living body, the object information acquisition apparatus according to this embodiment can perform imaging for a blood vessel which is contrasted by blood that serves as an optical absorption body present in the object 600. Also, the optical absorption body may be hemoglobin, water, melanin, collagen, lipid, or a living-body tissue formed of these, which has a relatively large optical absorption coefficient in the living body.

### Second Embodiment

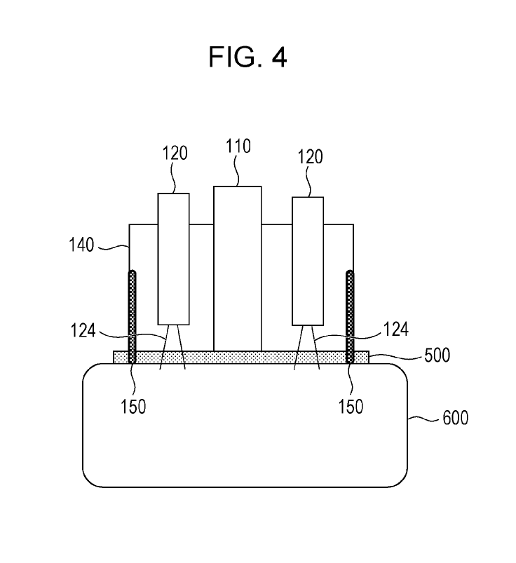

Next, a photoacoustic probe according to a second embodiment is described with reference to FIG. 4. FIG. 4 is a cross-sectional view of the photoacoustic probe according to this embodiment. This embodiment differs from the first embodiment in that the photoacoustic probe includes a smoothening member 150 that smoothens the acoustic matching member 500. The smoothening member is like a scraper.

An end portion of the smoothening member 150 is located closer to the object than the photoacoustic wave detection surface of the acoustic wave detector 110. With this configuration, when an operator performs scanning with the photoacoustic probe, the smoothening member 150 directly contacts the acoustic matching member 500, and hence smoothens the acoustic matching member 500. Since the acoustic matching member 500 is smoothened, likelihood of occurrence of that the acoustic matching member 500 is locally bulged can be reduced. Accordingly, likelihood of occurrence of that the locally bulged acoustic matching member 500 adheres to the exit surface of the optical system 120 can be reduced.

The smoothening member 150 may be arranged in any form as long as an end portion of the smoothening member 150 is located closer to the object 600 than the photoacoustic wave detection surface of the acoustic wave detector 110.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2012-132132, filed Jun. 11, 2012, which is hereby incorporated by reference herein in its entirety.

What is claimed is:

1. An object information acquisition apparatus, comprising:
an optical system configured to guide light that is emitted by a light source, and irradiate an object with the light; and
an acoustic wave detector configured to detect a photoacoustic wave that is generated when the object is irradiated with the light, and output a detection signal,
wherein the optical system and the acoustic wave detector are closely arranged, and
wherein, when the acoustic wave detector and the object acoustically contact each other through an acoustic matching member, a light exit surface of the optical system is arranged so that the light exit surface does not directly contact the acoustic matching member.

2. The object information acquisition apparatus according to claim 1, wherein the optical system and the acoustic wave detector are arranged so that the light exit surface of the optical system is located farther from the object than a photoacoustic wave detection surface of the acoustic wave detector when the acoustic wave detector and the object acoustically contact each other through the acoustic matching member.

3. The object information acquisition apparatus according to claim 1, wherein the optical system and the acoustic wave detector are arranged so that the light exit surface of the optical system is located farther from the object than a photoacoustic wave detection surface of the acoustic wave detector by at least 1 mm.

4. The object information acquisition apparatus according to claim 1, further comprising a casing configured to house the optical system and the acoustic wave detector.

5. The object information acquisition apparatus according to claim 4, wherein the optical system and the acoustic wave detector are arranged so that the light exit surface of the optical system is located at an inner side of the casing with respect to the photoacoustic wave detection surface of the acoustic wave detector.

6. The object information acquisition apparatus according to claim 4, wherein a portion of the casing through which the light emitted from the optical system propagates is located farther from the object than the photoacoustic wave detection surface of the acoustic wave detector.

7. The object information acquisition apparatus according to claim 1, wherein the optical system and the acoustic wave detector are arranged so that the acoustic wave detector is not irradiated with the light emitted from the optical system.

8. The object information acquisition apparatus according to claim 1, wherein the acoustic wave detector is formed of a material that transmits the light with a wavelength emitted from the optical system.

9. The object information acquisition apparatus according to claim 1, further comprising a signal processing unit configured to acquire object information based on the detection signal.

10. The object information acquisition apparatus according to claim 1, further comprising a smoothening member configured to smoothen the acoustic matching member.

11. The object information acquisition apparatus according to claim 1, wherein the acoustic wave detector includes a capacitive transducer.

12. A photoacoustic probe, comprising:
an optical system configured to guide light that is emitted by a light source, and irradiate an object with the light;
an acoustic wave detector configured to detect a photoacoustic wave that is generated when the object is irradiated with the light, and output a detection signal; and
a casing configured to house the optical system and the acoustic wave detector,
wherein the optical system and the acoustic wave detector are closely arranged, and
wherein a light exit surface of the optical system is arranged so that the light exit surface is located at an inner side of the casing with respect to a photoacoustic wave detection surface of the acoustic wave detector.

13. The photoacoustic probe according to claim 12, wherein the optical system and the acoustic wave detector are arranged so that the acoustic wave detector is not irradiated with the light emitted from the optical system.

14. The photoacoustic probe according to claim 12, wherein the optical system and the acoustic wave detector are arranged so that the light exit surface of the optical system is located farther from the object than the photoacoustic wave detection surface of the acoustic wave detector by at least 1 mm.

15. The photoacoustic probe according to claim 12, wherein the acoustic wave detector is formed of a material that transmits the light with a wavelength emitted from the optical system.

16. The photoacoustic probe according to claim 12, wherein a portion of the casing through which the light emitted from the optical system propagates is located farther from the object than the photoacoustic wave detection surface of the acoustic wave detector.

17. The photoacoustic probe according to claim 12, further comprising a signal processing unit configured to acquire object information based on the detection signal.

18. The photoacoustic probe according to claim 12, wherein the acoustic wave detector includes a capacitive transducer.
